(19) United States
(12) Patent Application Publication
(10) Pub. No.: US 2011/0124748 A1
(43) Pub. Date: May 26, 2011

(54) Coal and Biomass Conversion to Multiple Cleaner Energy Solutions System producing Hydrogen, Synthetic Fuels, Oils and Lubricants, Substitute Natural Gas and Clean Electricity

(75) Inventor: Lai O. Kuku, Gilbert, AZ (US)

(73) Assignee: Mr. Lai O. kuku, Gilbert, AZ (US)

(21) Appl. No.: 12/858,414

(22) Filed: Aug. 17, 2010

Related U.S. Application Data
(60) Provisional application No. 61/234,982, filed on Aug. 18, 2009.

(51) Int. Cl. C07C 27/00; F02C 3/28; C10J 3/00; C01B 3/32; C01B 3/12

(52) U.S. Cl. 518/700; 60/781; 48/210; 48/197 R; 48/127.5; 423/655

(57) ABSTRACT

The system contained within this application for patent protection provides the ability to produce clean syngas, natural gas, synthetic fuels, electricity, hydrogen fuels, and oil substitutes using a variety of materials. These materials include, but are not limited to: coal, biomass (including but not limited to municipal solid wastes), and agricultural byproducts. The fuels and electricity generated by this system can immediately be utilized by existing power and transportation grids, and as such, allow for rapid integration into the nation's energy needs. The system also removes and sequesters carbon dioxide, creating a clean, environmentally responsible supply of multiple types of power. The overall process provides an alternative to current oil and power solutions, allowing for domestic production of various energy requirements, creating the possibility for the reduced dependence on foreign imports for energy needs.

### I. INTRODUCTION

This Non-Provisional Utility Patent application claim benefits of a previously filed Provisional Patent Application #61/234,982, filed Aug. 18, 2009 by Lai O. Kuku, as defined by the United States Patent and Trademark Office (USTPO). Millenium SynthFuels is an alternative fuel and energy venture, created with the goal of providing the United States of America with the ability to produce alternative renewable energy and synthetic fuels domestically. The information contained within this application is proprietary, including but not limited to processes, drawings/schematics, and research. As such, this application seeks patent protection for said information under United States Patent laws. The general purpose of this application is to patent the processes and systems used for multiple types of energy conversion utilizing coal and biomass. Where required, separate forms will be attached to this document in accordance with patent application guidelines set forth by the USTPO on their web site.

### II. BRIEF DESCRIPTION OF BACKGROUND, PROCESS, AND DRAWINGS

### a) Background of Process

Millenium SynthFuels has created a new concept in alternative fuels and renewable energy. The intent is to build plants in the United States and other global locations that utilize a combination of coal and biomass as feedstock that will be converted into multiple forms of renewable energy within individual, stand-alone plants. No alternative energy research, development, proposal, or functioning plant currently exists anywhere that demonstrates or intends to demonstrate, a complete process flow for a multiple energy output plant. As this is the first of such alternative fuels and energy solutions, Millenium SynthFuels hereby tenders a Non-Provisional Utility United States Patent application to the United States Patent and Trademark Office for early receipt and confirmation, and also for examination and issue of the patent. Millenium SynthFuels claims rights to the overall process described herein, and requests United States Patent protection of the processes detailed herein, construction and operation of facilities designed to utilize the overall process contained herein, construction and operation of facilities designed to utilize the portions of the process to which specific claims are made within this document, and materials generated by any use of the processes and methods for which this patent application is being submitted.

### b) Summary of Process

The process flow includes the conversion of coal and lignocellulosic Biomass (i.e., Wood chips, pellets, plant residues, coconut husks, corn husks, tall grassy materials, and straw materials) into synthesis/biosynthesis gas (Syngas/Biosyngas). The Syngas/Biosyngas will be converted in one process flow into:
1. Hydrogen Fuels (Gas)
2. Substitute Natural Gas (SNG)
3. Fischer Tropsch Fuels and products (Sulfur free Synthetic Gasoline, Diesel, Jet fuels, Waxes, Paraffin, synthetic Oils, Lubricants, and Olefins)
4. Clean Electricity.

Complete details of the process flow have been included in the following pages of this documentation. All the aforementioned multiple energy solutions output of the Millenium SynthFuels' Coal and Biomass Conversion to Multiple Clean Energy Solutions System will incorporate carbon capture and sequestration, for the removal of pure CO₂ and acid gases including mercury oxides, sulfur oxides, and nitrous oxides as a part of the clean and complete energy solutions system.

### c) BRIEF DESCRIPTION OF DRAWINGS

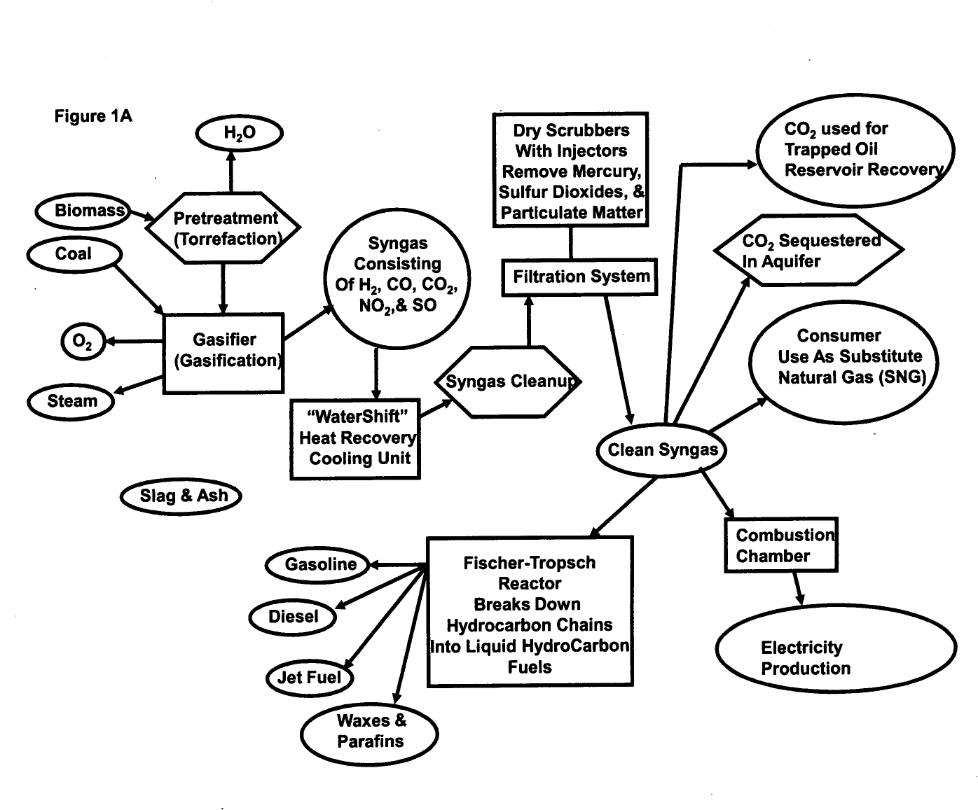

FIG. 1A is a broad overview of the entire Millenium SynthFuels process that this patent claim is being filed for. This drawing shows the material input, an overview of the entire process flow, and the product output made possible through the process.

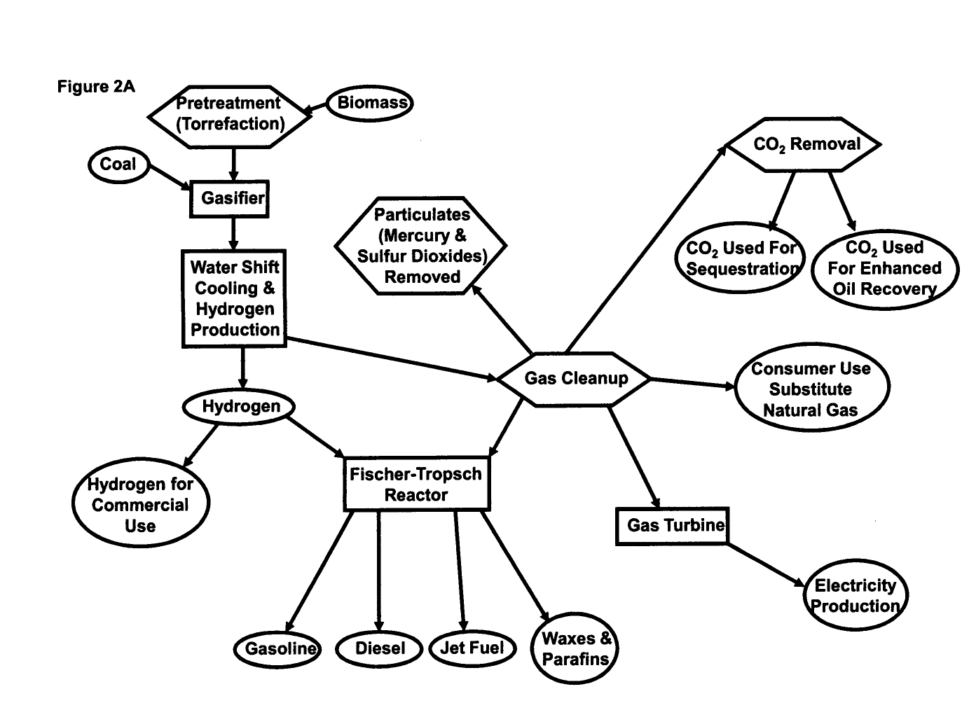

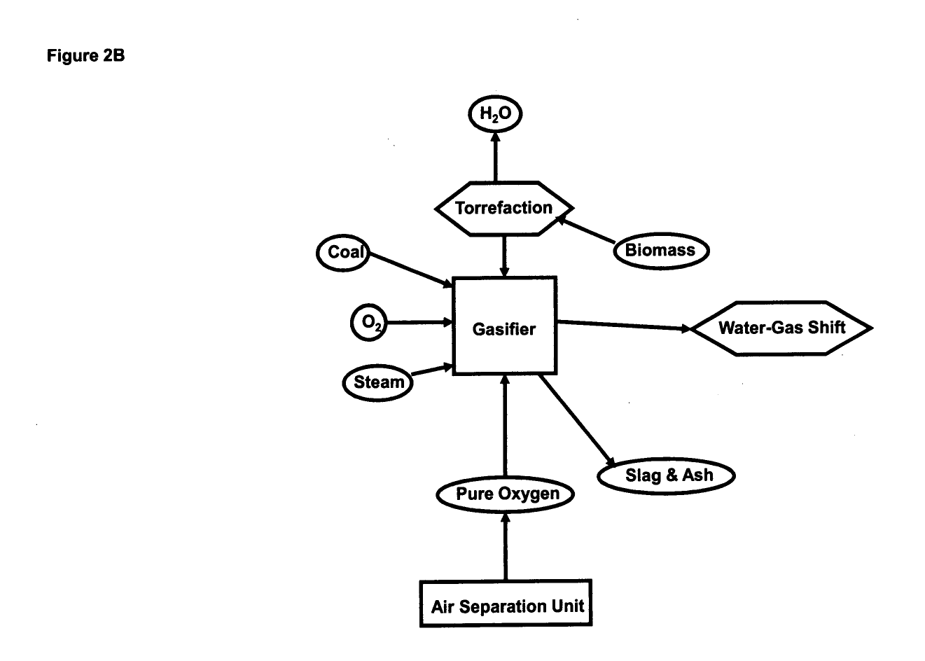

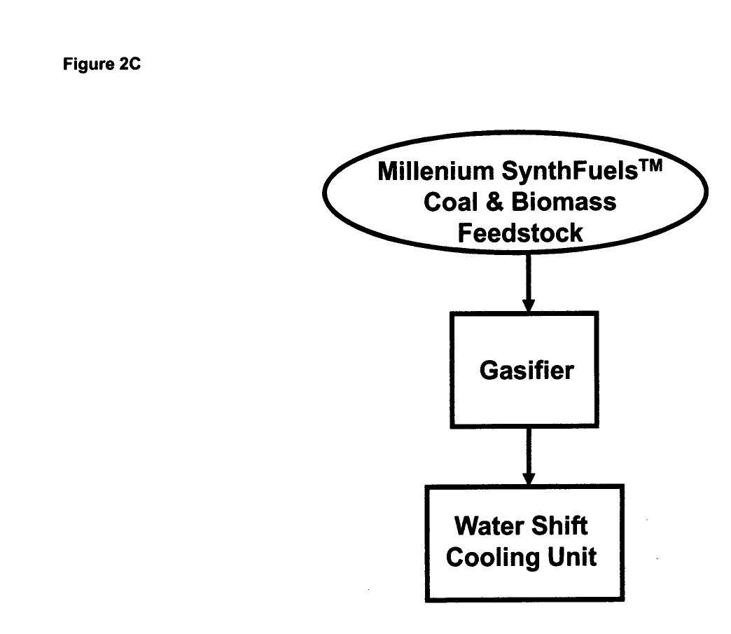

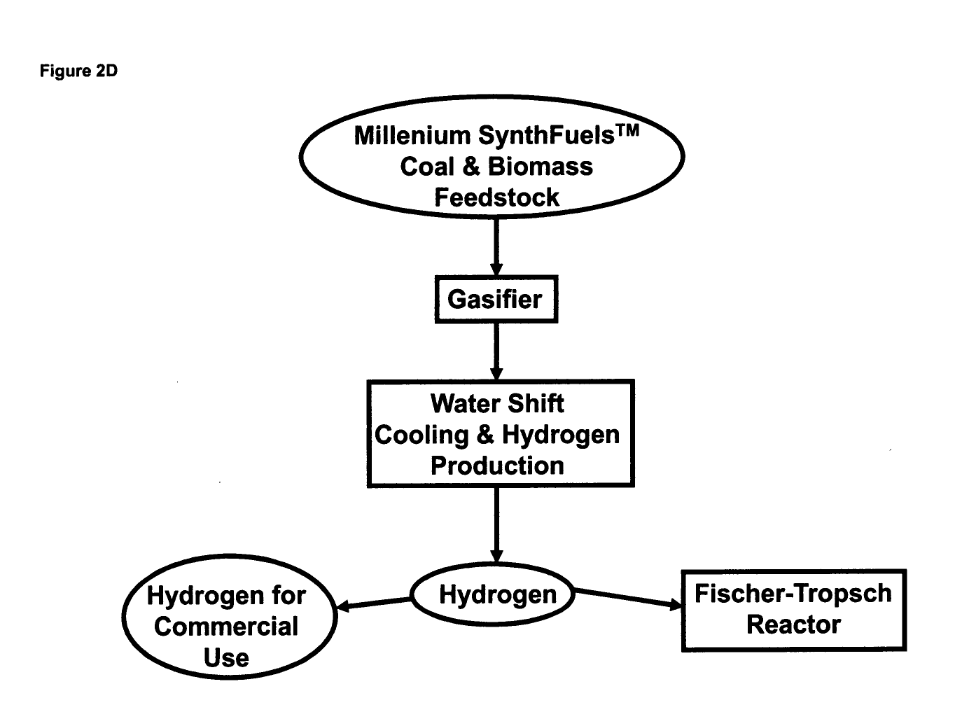

FIG. 2A demonstrates an overall process flow for materials through the Coal and Biomass Conversion to Multiple Clean Energy Solutions System. This figure begins with the feedstock, being either coal or biomass, with provisions for either. The materials enter into the gasifier to begin the process of separating constituent components. Upon exiting the gasifier, the output material is then processed through various stages that remove or further process the individual output components. FIG. 1A demonstrates the material output of Millenium SynthFuels process to be gasoline, diesel, jet fuel, waxes and paraffin, sequestered and/or captured CO₂, substitute natural gas, hydrogen, and electricity. It is important to note that FIG. 1A represents an individual, stand-alone plant being used to produce multiple forms of renewable energy.

FIG. 2B displays how the Millenium SynthFuels process will utilize both coal and biomass simultaneously for the gasification process. The coal and biomass graphics represent the feedstock, and the water droplet represents the water released in the torrefaction process, which removes moisture from the various biomass materials. The gasifier will use oxygen to enhance gasification. The air separation unit will produce and supply pure oxygen as required. The pieces of equipment in the drawing is the gasifier, air separation unit, and water gas shift, which demonstrates how material flows through the gasification process.

FIG. 2C demonstrates the transfer of material through the first three stages of the process. The first stage is the raw material, including the torrefied biomass, the second represents the gasification process, and the third demonstrates the transfer to the third step; that of the water shift cooling unit. The Water Gas Shift provides additional cooling of raw syngas exiting the gasifier, and also produces additional hydrogen.

FIG. 2D shows the flow of material demonstrated in FIG. 1C, moving beyond the water shift cooling unit and into the segregation of hydrogen from the material flow. The hydrogen removed can be utilized for commercial and industrial uses, and also for upgrading/hydrogenating synthetic fuels production. Reference is made of the use of the additional hydrogen production during the Fischer Tropsch synthesis phase.

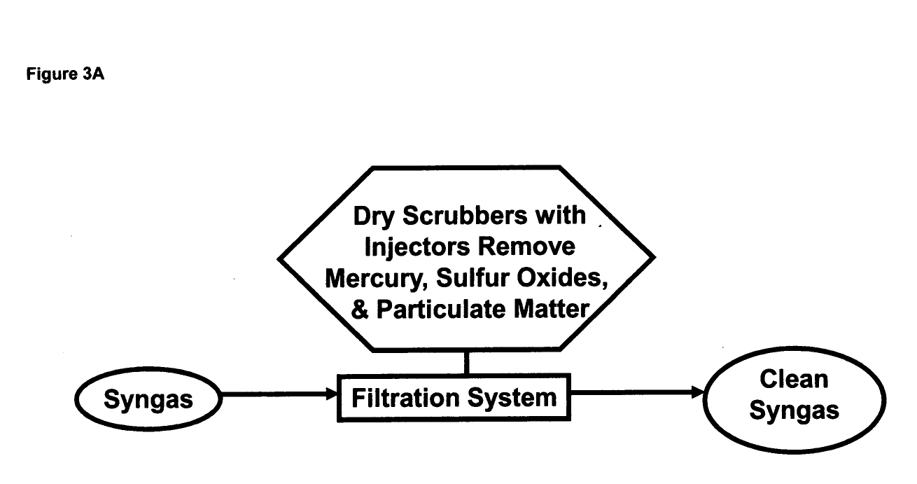

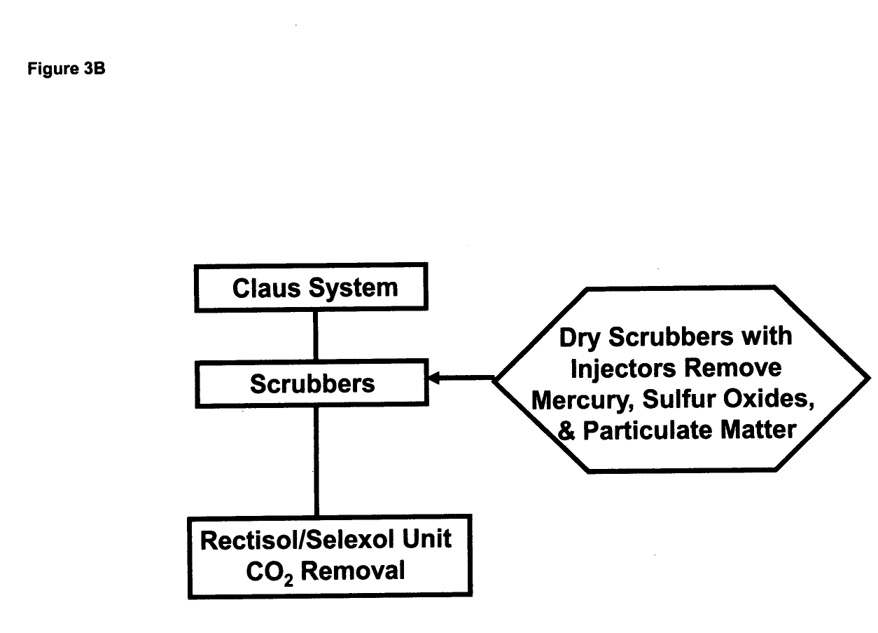

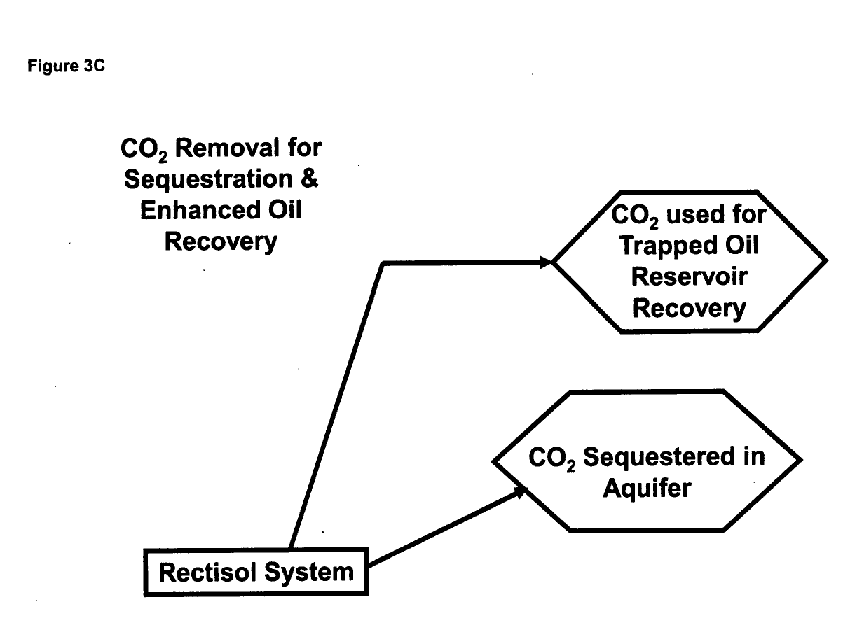

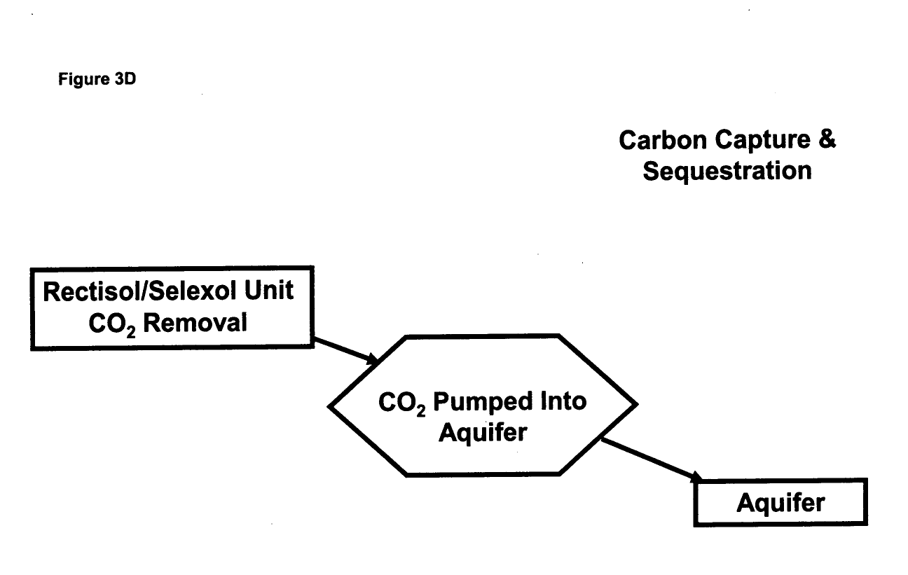

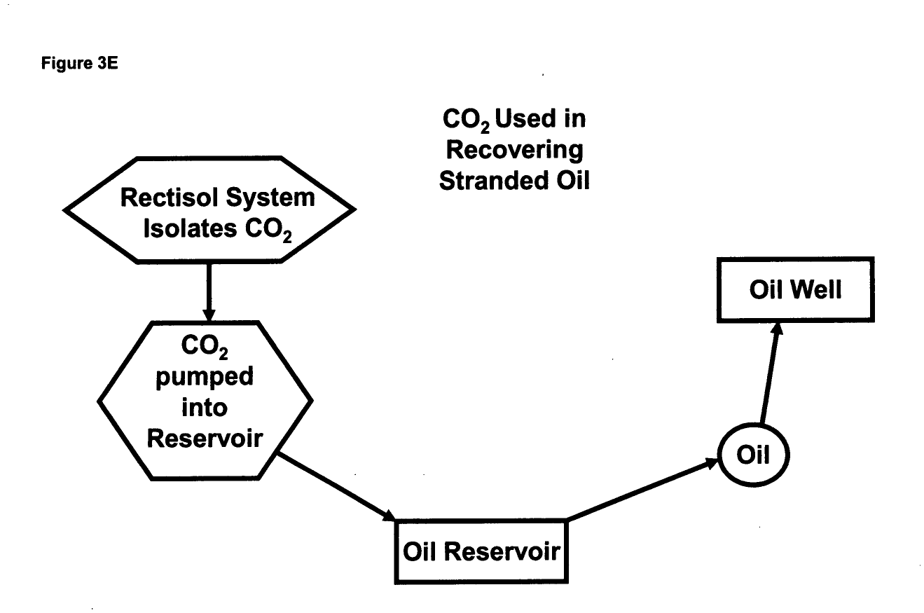

FIG. 3A demonstrates how the plant's Rectisol Filtration System will remove any hydrogen sulfides and carbon dioxide from the syngas material flow. Dry scrubbers with hydrated lime injectors will remove mercury and sulfur oxides and particulate matter from the syngas material flow. There are two potential destinations for the carbon dioxide; the first is the sequestration of the gas in underground aquifers, and the second pumping the gas into underground oil wells to recover oil stores.

FIG. 3B gives a closer look at the Rectisol Filtration System, including the Claus system, showing how the dry scrubber removes mercury, sulfur oxides, and particulate matter from the material flow. Sulfur oxide removed will be sent to a Claus system, which will convert the sulfur oxide into elemental sulfur, which can be used in the production of commercial grade fertilizer.

FIG. 3C demonstrates how the plant's Rectisol Filtration System will remove and sequester carbon dioxide from the material flow. There are two potential destinations for the carbon dioxide; the first is the sequestration of the gas in underground saline aquifers (FIG. 3D), and the second pumping the gas into underground oil wells to recover oil stores (FIG. 3E).

FIG. 3D shows the process of carbon dioxide removed and captured from the syngas stream by the Rectisol unit being pumped into underground saline aquifers for sequestration. This is the process of carbon capture and storage or sequestration.

FIG. 3E shows the use of carbon dioxide captured from the Rectisol unit being pumped into oil wells to recover stranded oil. Over 11 billion metric tons of carbon dioxide is required to recover 400 billion barrels of stranded United States domestic crude oil.

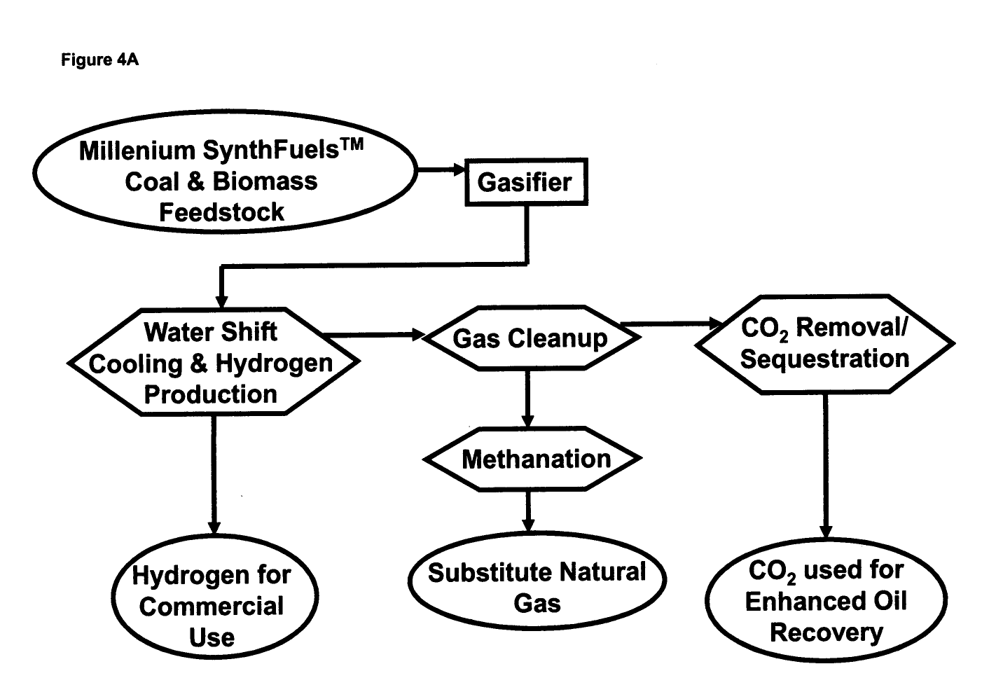

FIG. 4A demonstrates the material flow through the process that allows for the production of Syngas that can be used as a substitute for natural gas, also showing carbon capture and hydrogen segregation at the same stage in the process.

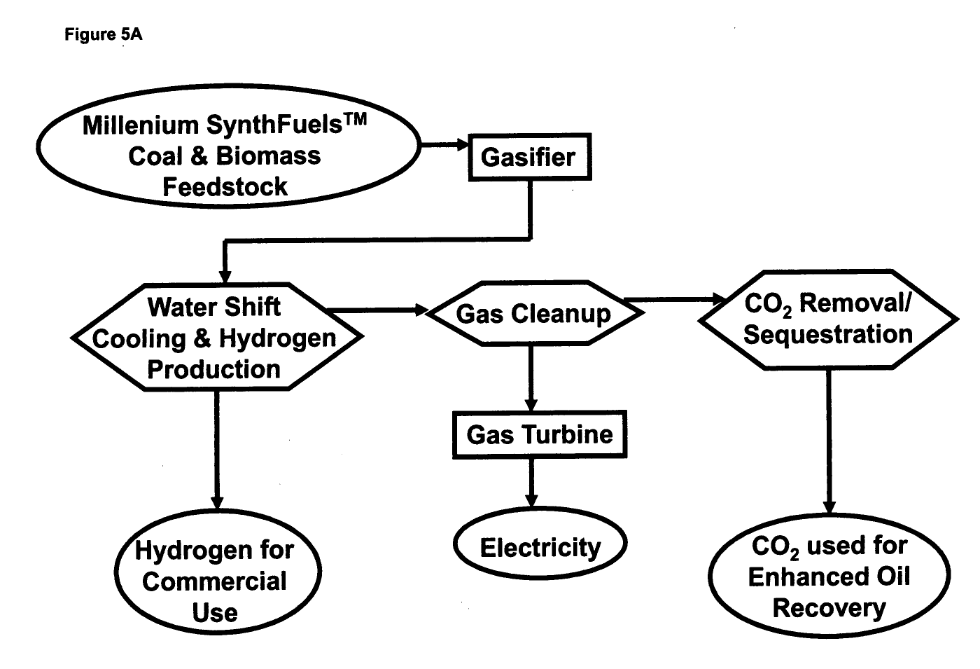

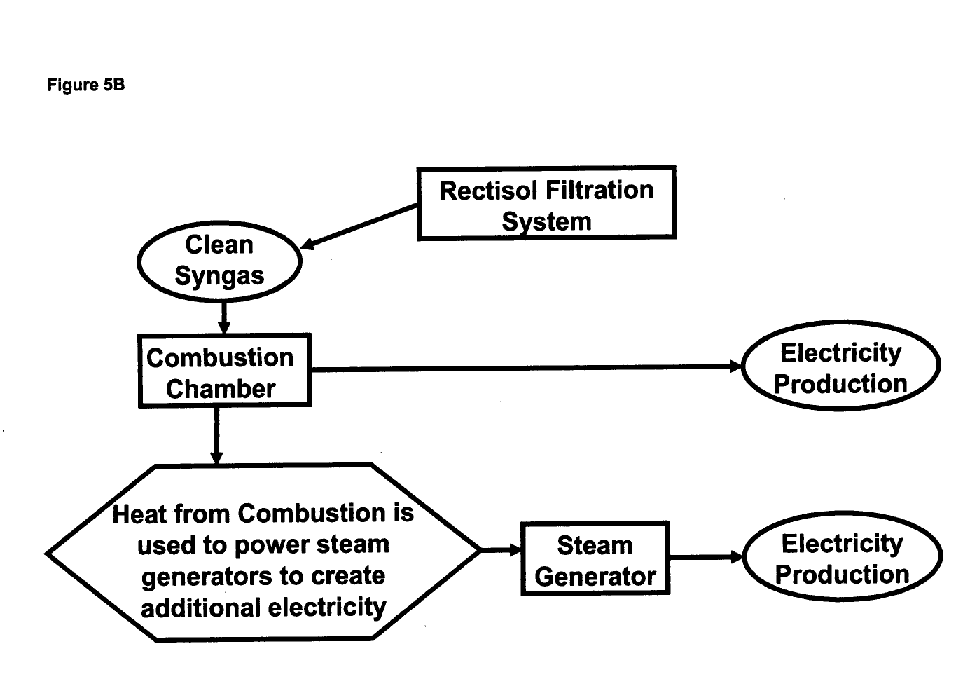

FIG. 5A displays the process flow to the stage of electricity production, running concurrently with the production of syngas, hydrogen, and the carbon capture process.

FIG. 5B describes in greater detail how electricity will be produced as a result of the material processing by utilizing the syngas produced to be combusted in combustion chamber to run gas turbines for electricity, and heat created in the process will be used to heat water for steam turbines for additional electricity production.

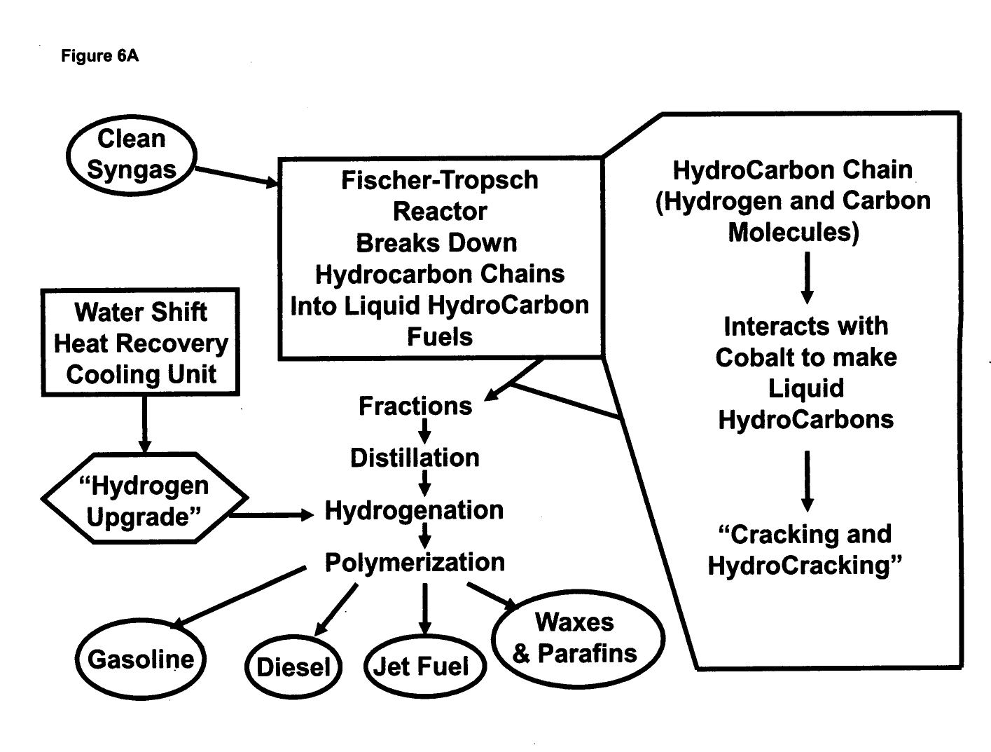

FIG. 6A is a breakdown of how the Fischer-Tropsch (FT) Synthesis Process works, and also displays the output capabilities of the process. It demonstrates the processes effect on hydrocarbon chains, altering their physical properties by converting them into liquid form. Once liquefied, these hydrocarbons are ‘cracked’, distilled, and converted into the various ultra clean synthetic fuels output forms.

### III. SPECIFICATION

### a) Title of Invention

Coal & Biomass Conversion to Multiple Cleaner Energy Solutions System producing Hydrogen, Synthetic Fuels, Oils and Lubricants, Substitute Natural Gas and Clean Electricity

### b) Description of the Preferred Embodiment

### 1.0 Millenium SynthFuels Coal & Biomass Conversion to Multiple Cleaner Energy Solutions System Preamble

In the United States, Coal to Liquids (CTL) technology is still at the research and development stage, with no entity possessing the ability to utilize these technologies on a commercial scale. The entities that are currently engaged in the research and development of these technologies have focused solely on CTL or Biomass to Liquids (BTL), primarily developing technologies for liquid fuel solutions rather than broad spectrum energy generation capabilities. In addition, only one electricity plant in the United States has been converted with provisions for reducing carbon dioxide emissions. Millenium SynthFuels has created a different solution in the field of alternative fuels and renewable energy. Our goal is to build plants in the United States and other global locations that utilize a combination of coal and biomass as feedstock, which will be converted to provide multiple renewable energy solutions within individual plants. No alternative energy research, development, proposal, or functioning plant currently exists anywhere that demonstrates or intends to demonstrate, a complete process flow for a multiple energy output plant. Millenium SynthFuels hereby tenders the Coal and Biomass Conversion to Multiple Clean Energy Solutions System for United States Patent protection. FIG. 1A depicts an overview of the complete process.

Claim #1

Millenium SynthFuels claims rights to the process described herein as an overall process flow, referred to as the Millenium SynthFuels Coal & Biomass Conversion to Multiple Cleaner Energy Solutions System.

### 2.0 Coal and Biomass Conversion to Multiple Cleaner Energy Solutions System

FIG. 2A shows the complete process flow of the proposed Millenium SynthFuels Coal and Biomass Conversion to Multiple Cleaner Energy Solutions System. The first stage of the process is the co-gasification of coal and lignocellulosic biomass into synthesis/biosynthesis gas, which will be processed into multiple clean energy solutions.

### 3.0 Coal and Biomass Conversion to Synthesis Gas

3.1 Torrefaction Pretreatment

This process involves the integration of both coal and biomass feedstock in the gasification stage. Biomass feedstock such as wood chips, plant residue, and most straw and grass like materials have a high moisture content which must be pretreated before gasification. The pretreatment is with a partial oxygenation process known as torrefaction. During torrefaction, the biomass materials are slowly heated to a range of 200-300° C., without oxygen, and at normal atmospheric pressure. The torrefied biomass has many improved properties for gasification, including higher energy density, higher energy efficiency, suitability for different fuel applications, and a much lower moisture content than the initial biomass material. Torrefaction also helps compacts loose strands of biomass into pellet sizes which enables easier handling and gasifier loading. Consequently, torrefaction increases the conversion efficiency of biomass to syngas (Biosyngas). As such, the torrefied biomass can be easily blended or alternated with coal in gasifiers for the production of synthesis gas. Once treated, the coal and biomass can be introduced into an oxygen-blown entrained flow gasifier, along with steam. As it is in the case with coal gasification, the same process of pyrolysis, combustion, and gasification occurs during biomass gasification.

3.2 Coal and Biomass Co-Gasification

Claim #2

The Co-Gasification of coal and biomass (through the utilization of pretreatment and torrefaction) is unique to the Millenium SynthFuels process, and as such Millenium SynthFuels claims rights to the process. Coal and biomass are fed into the gasifier as displayed in FIG. 2B under pressure between 40 and 72 bars, dependent upon the gasifier type and capacity. During pyrolysis, carbonaceous particles of coal and biomass heat up producing char in the presence of oxygen produced by the air separation unit. The thermochemical reactions continue with combustion, which occurs when the released char reacts with oxygen creating carbon monoxide and carbon dioxide. Further, as char continues to reacts with carbon dioxide and steam, carbon monoxide and hydrogen are generated under high pressure and temperatures exceeding 1600° F. to complete the gasification process. A quench system built into the top of the gasifier provides initial cooling of the raw syngas before exiting the gasifier.

### 4.0 Syngas Cooling and Hydrogen Production

A built-in quench system reduces syngas temperature as it exits the gasifier. A water/gas shift is used for additional cooling and also to attain a balanced mixture of gases. Consequently, carbon monoxide in the raw syngas undergoes a chemical reaction with water creating additional carbon dioxide and hydrogen, as displayed in FIG. 2D.

Claim #3

Commercial hydrogen production through the method described herein is unique to this process, and as such, Millenium SynthFuels claims rights to the process. The hydrogen produced can be utilized for many products, including the reforming of methane, and the upgrade or hydrogenation of Fischer-Tropsch (FT) synthetic fuels. The Millenium SynthFuels Coal and Biomass Conversion to Cleaner Energy Solutions System incorporated within the process flow the utilization of hydrogen generated in the water/gas shift for the hydrogenation upgrade of FT synthetic fuels. In addition, this process will also serve as the hydrogen gas source that will be piped and sold for commercial use in hydrogen fuel cells. This is one of the clean energy solutions of the clean energy solutions process flow.

### 5.0 Syngas Cleanup

Synthesis gas (Syngas) is a combination of hydrogen, carbon monoxide, carbon dioxide and various acid gases, including sulfur and mercury oxides. With CO₂ concentration stated at 326 ppm of total carbon emissions, CO₂ and acid gas emissions must be addressed. Millenium SynthFuels' clean coal and biomass conversion process incorporates many technologies within plant design to prevent acid gas emissions, and the capture of over 95% of pure CO₂ emissions as FIG. 3A shows.

Claim #4

Removal of acid gases including nitrogen oxide, sulfur oxide, mercury oxide, particulate matter, and carbon dioxide through the method described herein is unique to this process, and as such, Millenium SynthFuels claims rights to the process.

As syngas leaves the gasifier, it is sent through a gas conditioner water shift, which cools the gas temperature and also generates additional hydrogen and carbon dioxide. The stream of cooled raw syngas contains carbon dioxide, carbon dioxide, mercury, and sulfur and nitrogen oxides. As the desired syngas composition is hydrogen and carbon monoxide, carbon dioxide, other acid gases and particulate matter must be removed. The Rectisol system will be used to address carbon dioxide emissions. The Rectisol system is a physical acid gas removal process that utilizes cold methanol as a solvent in removing hydrogen sulfide and carbon dioxide in separate streams from raw syngas. This process enables hydrogen sulfide to be piped to the Claus system for processing, in addition to the capture of pure CO₂, which will be further sequestered in aquifers or used in enhanced Oil recovery (EOR). In addition, Heat Recovery Steam Generators (HRSG) with a Selective Catalytic Reduction system which is ideal for heat recovery in combined cycle plants can also be utilized in controlling nitrogen oxide emissions.

Mercury and sulfur oxides are controlled with the use of dry scrubbing or dry sorbent injectors system, demonstrated in FIG. 3B as a Rectisol Unit with an attached Claus System and additional scrubbers. The dry sorbent injectors introduce limestone or hydrated lime into the gas stream, which causes a reaction with sulfur and mercury oxides. In addition, the dry scrubbers also provide particulate matter control by removing particulate matter in the stream of syngas. Once CO₂, SO₂, NO and other acid gases are removed, the clean stream of syngas can be upgraded as substitute natural gas (SNG), as the gas fuel for renewable liquid FT fuels, and in the generation of clean electricity.

Carbon Capture and Sequestration (CCS), also known as Carbon Capture and Storage, is the process for storing CO₂ at depth under the earth's surface. As demonstrated in FIG. 3C, the Rectisol is a physical acid gas removal process that utilizes cold methanol as a solvent within a separate stream to remove carbon dioxide from streams of raw syngas/Biosyngas. Once carbon dioxide has been removed, the gas is piped in a stream to be injected into depleted oil wells or as in FIG. 3C, sequestered in underground saline aquifers as carbon capture and sequestration (CCS). The CCS process begins with continuous injection of CO2 under pressure into the injection wells to depths of 6000 feet and greater. As pressure increases with depth, the increased pressure causes the injected CO2 to liquefy. As liquid CO2 in introduced into saline water, it dissolves. This is comparable to adding lime into a glass of water. The result is a change in the acidity of the saline water, which becomes heavier and sinks to the bottom of the saline formation, enters the pores of the rocks in the formation. This creates a reaction with the minerals in the saline rock formation and causes the CO2 to bind with the rock, and eventually creates new rock formations, trapping the CO2 indefinitely. As saline formations contain aquifers and permeable rock, the liquid CO2 also is absorbed and trapped in the pores of permeable rock, and becomes trapped as well. The impermeable rock above the permeable rock formations will keep the liquid CO2 trapped indefinitely as well. While another CO2 storage option includes un-useable coal seams as well as the aforementioned coal and gas reservoirs, the main question is will CO2 be trapped and for how long. The answer is yes, and with numerous studies and reports from CO2 Enhanced Oil Recovery (EOR), and Gas Recovery (EGR), almost 99-100% of the injected CO2 should remain captured or sequestered for well over 1100 years.

### 6.0 Clean Syngas as Substitute Natural Gas (SNG)

Claim #5

The production of Substitute Natural Gas through the method described herein is unique to this process, and as such, Millenium SynthFuels claims rights to the process.

As demonstrated by FIG. 4A, the process described herein has the capability to produce natural gas. Natural gas carries energy that can produce both heat and power. In addition, natural gas can be substituted for syngas in the production of clean electricity and FT liquids. Syngas/Biosyngas has similar properties to natural gas in energy potential and in the production of heat and power. Once acid gases have been scrubbed and CO₂ removed, clean syngas can undergo methanation, and gas conditioning for syngas to be piped into gas grids for commercial, industrial and residential use. Coal gasification in the production of substitute natural gas is referred to as SNG. As biomass will be used in conjunction with coal, the biomass to substitute natural gas produced is easily converted with the same energy carriage as natural gas. Biomass to SNG is known as BioSNG.

### 7.0 Clean Electricity

Claim #6

Amplified clean electricity with a combined gas turbine electric generation and captured heat to run additional steam generators for combined syngas to electricity and steam electricity generation. Through the method described herein is unique to this process, and as such, Millenium SynthFuels claims rights to the process.

As FIG. 5A and 5B illustrate, clean syngas leaving the Rectisol system is piped to a combustor turbine and used to power gas turbines and generators in producing clean electricity. Electric power produced can be amplified by recovering combustion and turbine exhaust gases through heat exchangers to run steam turbines for additional electric power generation. As 50% of the 800+GW of electric power required daily in the United States and other global locations are produced by coal fired plants, the Millenium SynthFuels Coal and Biomass Conversion to Multiple Cleaner Energy Solution process flow can be utilized to convert existing coal fired plants into combined cycle power plants. The only requirement is the installation of gasifiers; syngas cleanup and CO₂ capture equipment, and the implementation of a combined gas steam turbine electricity generation system. In essence, any existing coal fired plant could be converted into a complete energy solutions plant utilizing the processes which this application is claiming rights to.

### 8.0 Fischer-Tropsch (FT) Synthesis Process

Claim #7

The production of a variety of hydrocarbon synthetic fuels including higher octane gasoline, higher cetane synthetic diesel, synthetic oils and lubricants and synthetic jet fuel as combined synthetic fuels and products, all from a singular process flow, through the method described herein is unique to this process, and as such, Millenium SynthFuels claims rights to the process.

In the FT process, demonstrated in FIG. 6A, the hydrogen and carbon monoxide in the syngas undergoes a catalyzed chemical reaction with a Cobalt, Iron, or Nickel based catalyst. In a cobalt based reactor, hydrogen and carbon monoxide in syngas undergoes a catalyzed chemical reaction with the cobalt, and becomes converted to different liquid hydrocarbons. More processing is involved before the raw hydrocarbon liquids can be classified as fuels. Part of the additional process includes ‘cracking’, which breaks down the weighted hydrocarbon liquid chains into lighter hydrocarbon chain liquids. In addition, the hydrocarbon liquids undergo ‘hydro-cracking’, which involves the introduction of pressurized hydrogen and a catalyst. The process creates a reaction that changes and breaks down liquid hydrocarbon chains and purifies the hydrocarbon liquids from sulfur and nitrogen. This is the process that creates naphtha liquids, including gasoline, diesel, and jet fuel. The liquid fuels are further polymerized, hydrogenised, fractionated, and distilled in creating higher octane/cetane nitrogen free and ultralow sulfur/sulfur-free liquid fuels including gasoline, diesel, and synthetic jet fuels, making these fuels “Ultra Clean” clean-burning.

1. Millenium SynthFuels claims rights to the process described herein as an overall process flow, referred to as the Millenium SynthFuels Coal & Biomass Conversion to Multiple Cleaner Energy Solutions System.

2. The Co-Gasification of coal and biomass (through the utilization of pretreatment and torrefaction) is unique to the Millenium SynthFuels process, and as such Millenium SynthFuels claims rights to the process.

3. Commercial hydrogen production through the method described herein is unique to this process, and as such, Millenium SynthFuels claims rights to the process.

4. Removal of acid gases including nitrogen oxide, sulfur oxide, mercury oxide, particulate matter, and carbon dioxide through the method described herein is unique to this process, and as such, Millenium SynthFuels claims rights to the process.

5. The production of Substitute Natural Gas through the method described herein is unique to this process, and as such, Millenium SynthFuels claims rights to the process.

6. Amplified clean electricity with a combined gas turbine electric generation and captured heat to run additional steam generators for combined syngas to electricity and steam electricity generation. Through the method described herein is unique to this process, and as such, Millenium SynthFuels claims rights to the process.

7. The production of a variety of hydrocarbon synthetic fuels including higher octane gasoline, higher cetane synthetic diesel, synthetic oils and lubricants and synthetic jet fuel as combined synthetic fuels and products, all from a singular process flow, through the method described herein is unique to this process, and as such, Millenium SynthFuels claims rights to the process.
